(19) United States
(12) Patent Application Publication
(10) Pub. No.: US 2026/0021169 A1
(43) Pub. Date: Jan. 22, 2026

(54) CO-TRANSPLANTATION OF MMP-9 ENHANCED MESENCHYMAL STEM CELLS AND PANCREATIC ISLETS FOR THE TREATMENT OF DIABETES

(71) Applicant: University of Maryland, Baltimore, Baltimore, MD (US)

(72) Inventors: Raphael Meier, Baltimore, MD (US); Anjali Verma, Ellicott City, MD (US)

(21) Appl. No.: 19/274,537

(22) Filed: Jul. 19, 2025

Related U.S. Application Data
(60) Provisional application No. 63/673,302, filed on Jul. 19, 2024.

(51) Int. Cl. A61K 38/48; A61K 35/28; A61K 35/39; A61K 47/36; A61P 3/10

(52) U.S. Cl. CPC A61K 38/4886; A61K 35/28; A61K 35/39; A61K 47/36; A61P 3/10; C12Y 304/24035

(57) ABSTRACT

The present invention is directed to a cellular therapeutic composition for treating diabetes in a subject comprising a therapeutically effective amount of insulin producing cells; and a therapeutically effective amount of modified mesenchymal stem cells, wherein the mesenchymal stem cells have been modified to increase expression of matrix metalloproteinase-9 (MMP-9) or a fragment or variant thereof.

### CROSS-REFERENCE TO RELATED APPLICATIONS

This application claims the benefit of U.S. Appl. No. 63/673,302, filed on Jul. 19, 2024, which is incorporated by reference in its entirety.

### INCORPORATION-BY-REFERENCE OF MATERIAL SUBMITTED ELECTRONICALLY

Incorporated by reference in its entirety herein is a computer-readable sequence listing submitted concurrently herewith and identified as follows: One 9,231 Byte xml file named “Sequence_listing_xml,” created on Jul. 16, 2025.

### FIELD OF THE INVENTION

The field of the invention relates to medicine and therapeutics, in particular, cell therapies and therapeutics to treat diabetes.

### BACKGROUND OF THE INVENTION

Diabetes is one of the most prevalent diseases worldwide, expected to impact 629 million patients by 2045 (IFD, International Diabetes Federation, 2021; Wild, S., et al., Diabetes Care, (2004), 27:1047-53). In both children and adults, the hallmark of Type 1 diabetes (T1D) is the loss of insulin-secreting pancreatic β-cells. About 700,000 new cases are diagnosed each year (Incidence and trends of childhood Type 1 diabetes worldwide 1990-1999. Diabet Med, (2006), 23:857-66). The disease is hard on many organs, the odds ratio of developing kidney failure is particularly high.

T1D patients need exogenous insulin to maintain their blood sugar. While insulin therapy is essential for individuals with TID, it comes with its own set of challenges such as episodes of hypoglycemia, mealtime management, dosing accuracy, and severe long-term complications.

Pancreatic islet transplantation has the potential to restore physiological insulin secretion and to achieve better glycemic control than exogenous insulin alone. It is far less invasive than a whole pancreas transplant and virtually eliminates hypoglycemia unawareness episodes. On Jun. 28, 2023, the United States Food and Drug Administration (FDA) approved the first allogeneic pancreatic islet cell therapy for the treatment of TID. Currently, limited viability and poor engraftment of pancreatic islets after intraportal infusion drastically reduce the chances of insulin independence and limit its application.

It was shown that Mesenchymal Stem Cells (MSCs) can improve survival, engraftment and function of encapsulated pancreatic islets by cell-to-cell contact (Montanari, E., et al., Stem Cell Res Ther, (2017), 8:199). Matrix Metalloproteinase-9 (MMP-9), one of the key molecules secreted by MSCs, is essential for beta cell function and islet vascularization (Christoffersson, G., et al., The American Journal of Pathology, (2015), 185:1094-1103). The positive effect of MSCs on encapsulated islets has been reported (Davis, N. E., et al., Biomaterials, (2012), 33:6691-7). However, the magnitude of the effect was modest.

What is needed are new and effective therapies to treat or cure diabetes, including TID. This background information is provided for informational purposes only. No admission is necessarily intended, nor should it be construed, that any of the preceding information constitutes prior art against the present invention.

### SUMMARY OF THE INVENTION

It is to be understood that both the foregoing general description of the embodiments and the following detailed description are exemplary, and thus do not restrict the scope of the embodiments.

The present invention is directed to a novel cell therapy involving co-encapsulation of MMP9-enhanced MSC and pancreatic islets to treat T1D patients. The present disclosure provides that function and vascularization can be further improved by specifically increasing MMP-9 levels in MSC cells. It is shown herein that modified MSCs enhance the secretion of MMP-9 over several fold. The cell therapeutic is based on the transplantation of co-encapsulated MMP-9-secreting MSCs and pancreatic islets for the successful treatment of diabetes, including TID.

In one aspect, the invention provides a cellular therapeutic composition for treating diabetes in a subject comprising:
i) a therapeutically effective amount of insulin producing cells; and
ii) a therapeutically effective amount of modified mesenchymal stem cells, wherein the mesenchymal stem cells have been modified to increase expression of matrix metalloproteinase-9 (MMP-9) or a fragment or variant thereof.

In some embodiments, the insulin producing cells are pancreatic cells.

In some embodiments, the cellular therapeutic composition of claim 2, wherein the pancreatic cells are islet cells. In some embodiments, the islet cells are β-cells.

In some embodiments, the insulin producing cells are differentiated from stem cells. In some embodiments, the stem cells are induced pluripotent stem (iPS) cells. In some embodiments, the stem cells are allogenic. In some embodiments, the stem cells are autologous.

In some embodiments, the insulin producing cells are capable of secreting an effective amount of insulin when administered to the subject, thereby lowering blood sugar levels in the subject and treating diabetes.

In some embodiments, the mesenchymal stem cells are allogenic. In some embodiments, the mesenchymal stem cells are autologous. In some embodiments, the mesenchymal stem cells are derived from bone marrow.

In some embodiments, the mesenchymal stem cells have been modified to increase expression of MMP-9 by administering to the mesenchymal stem cells a nucleic acid that encodes MMP-9 or a fragment or variant thereof. In some embodiments, the nucleic acid is provided by a viral vector. In some embodiments, the viral vector is a lentiviral vector.

In some embodiments, the mesenchymal stem cells have been modified to increase expression of MMP-9 by reducing expression of SMAD4. In some embodiments, the SMAD4 expression is reduced by administering to the mesenchymal stem cells a CRISPR-Cas9 system. In some embodiments, the SMAD4 expression is reduced by administering to the mesenchymal stem cells a nucleic acid. In some embodiments, the nucleic acid is a siRNA.

In some embodiments, the mesenchymal stem cells have been modified to increase expression of MMP-9 by administering to the mesenchymal stem cells a CRISPRa/dCas9 and sgRNA that binds to an upstream promoter region of MMP-9.

In some embodiments, the insulin producing cells and modified mesenchymal stem cells are co-encapsulated. In some embodiments, the encapsulated cells are immobilized within a polymeric semi-permeable scaffold.

In some embodiments, the insulin producing cells and modified mesenchymal stem cells are encapsulated by a semi-permeable scaffold comprising alginate. In some embodiments, the semi-permeable scaffold comprises calcium and alginate.

In some embodiments, the insulin producing cells and modified mesenchymal stem cells are co-encapsulated in calcium-alginate microbeads of about 550 μm diameter.

In another aspect, the invention provides a method of treating or preventing diabetes in a subject, comprising administering to the subject an effective amount of the cellular therapeutic composition herein. In some embodiments, the subject has Type I diabetes. In some embodiments, the subject has Type II diabetes.

In some embodiments, the cellular therapeutic composition is administered to the subject by infusion through the subject's portal vein. In some embodiments, the cellular therapeutic composition is administered to the subject intraperitoneally. In some embodiments, the insulin producing cells are engrafted in the subject following administration. In some embodiments, the insulin producing cells secrete an effective amount of insulin in response to the subject's blood glucose levels, thereby lowering blood glucose levels in the subject and treating diabetes.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE FIGURES

The skilled artisan will understand that the drawings, described below, are for illustration purposes only. The drawings are not intended to limit the scope of the present teachings in any way.

FIG. 1. mRNA expression of MMP-9 gene (a) SMAD4-silenced MSCs, (b) CRISPRa-modified MSCs, and (C) lentivirus-mediated MMP-9 overexpression. (d) Quantification of MMP-9 protein by modified MSCs compared to non-modified MSCs (only lentivirus MMP-9 are shown).

FIG. 2. (A) Encapsulated human islet alone. (B) Encapsulated human islet with MSCs. (C) FDA staining showing islet viability inside the calcium-alginate capsule.

FIG. 3. (A) Intimate contact in culture between MSCs (vimentin, green) and islets: β-cells (insulin, blue) and a-cells (glucagon, red). (B) Effect of MSCs on human islet insulin secretion (doubled). (C) Human islet graft survival in mice, free islets vs. encapsulated islets vs. encapsulated islets with MSCs (no immunosuppression).

FIG. 4. MSCs and MMP-9-MSCs promote increased insulin secretion in response to glucose. We performed a Glucose-induced insulin secretion (GSIS) to test the release of insulin following pancreatic beta cells (Rat insulinoma INS-1 cells) stimulation by high glucose levels. Insulin measure by ELISA is show. MSCs were cultured in transwells with rat insulinoma INS-1 beta cells. Highest amounts of insulin were observed in beta cells cultured with MMP-9 modified cells.

FIG. 5. Role of MSCs in diabetes and islet survival.

FIG. 6. Human islet isolation.

FIG. 7. (A) Encapsulation of Islets, Islets+MSCs, Islets+GM-MSC1, and islets+GM-MSC2. FDA/PI staining indicates cell viability after encapsulation. Both islet and MSCs survived encapsulation (green) with minimal cell death (red). (B) Glucose Stimulated Insulin Secretion by human islets when treated with indicated groups in low (2.8 mM) and high glucose (28 mM) condition. (C) Mice were transplanted with encapsulated islets (n=2), encapsulated islets+MSCs (n=2), encapsulated islets+GM-MSC1 (n=4), and islets+GM-MSC2 (n=2). Glycemia was measured regularly thereafter. Dashed black line represents Blood Glucose=360 mg/dL. Blue line, glycemia measurements in mice transplanted with encapsulated islets alone; red line, glycemia measurements in mice transplanted with encapsulated islets+MSCs; green line, glucose measurements in mice transplanted with encapsulated islets+GM-MSC1; purple line, glycemia measurements in mice transplanted with encapsulated islets+GM-MSC2. (D) Kaplan-Meier curve representing the percent xenograft survival. Blue line, the encapsulated islets alone; red line, the encapsulated islets+MSCs; green line, the encapsulated islets+GM-MSC1; purple line, the encapsulated islets+GM-MSC2.

### DETAILED DESCRIPTION

Reference will now be made in detail to the presently preferred embodiments of the invention which, together with the drawings and the following examples, serve to explain the principles of the invention. These embodiments describe in sufficient detail to enable those skilled in the art to practice the invention, and it is understood that other embodiments may be utilized, and that structural, biological, and chemical changes may be made without departing from the spirit and scope of the present invention. Unless defined otherwise, all technical and scientific terms used herein have the same meanings as commonly understood by one of ordinary skill in the art.

The practice of the present invention employs, unless otherwise indicated, conventional techniques of molecular biology (including recombinant techniques), microbiology, cell biology, biochemistry and immunology, which are within the skill of the art. Such techniques are explained fully in the literature. See, e.g., Sambrook et al. Molecular Cloning: A Laboratory Manual, 2^{nd} edition (1989); Current Protocols in Molecular Biology (F. M. Ausubel et al. eds. (1987)); the series Methods in Enzymology (Academic Press, Inc.); PCR: A Practical Approach (M. MacPherson et al. IRL Press at Oxford University Press (1991)); PCR 2: A Practical Approach (M. J. MacPherson, B. D. Hames and G. R. Taylor eds. (1995)); Antibodies, A Laboratory Manual (Harlow and Lane eds. (1988)); Using Antibodies, A Laboratory Manual (Harlow and Lane eds. (1999)); and Animal Cell Culture (R. I. Freshney ed. (1987)).

Definitions of common terms in molecular biology may be found, for example, in Benjamin Lewin, Genes VII, published by Oxford University Press, 2000 (ISBN 019879276X); Kendrew et al. (eds.); The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341).

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). The use of “or” means “and/or” unless stated otherwise. As used in the specification and claims, the singular form “a,” “an” and “the” include plural references unless the context clearly dictates otherwise. For example, the term “a cell” includes a plurality of cells, including mixtures thereof. The use of “comprise,” “comprises,” “comprising,” “include,” “includes,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of.”

As used herein, the term “about” means plus or minus 10% of the numerical value of the number with which it is being used.

The terms “nucleic acid,” and “polynucleotide,” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can also encompass analogues of natural nucleotides, as well as nucleotides that are modified in the base, sugar and/or phosphate moieties.

The terms “polypeptide,” “peptide” and “protein” are used interchangeably to refer to a polymer of amino acid residues. The term also applies to amino acid polymers in which one or more amino acids are chemical analogues or modified derivatives of corresponding naturally-occurring amino acids. The term “sequence” relates to a nucleotide sequence of any length, which can be DNA or RNA; can be linear, circular or branched and can be either single-stranded or double stranded.

The term “identity” relates to an exact nucleotide-to-nucleotide or amino acid-to-amino acid correspondence of two polynucleotides or polypeptide sequences, respectively. Two or more sequences (polynucleotide or amino acid) can be compared by determining their percent identity. Calculations of homology or sequence identity between two sequences (the terms are used interchangeably herein) are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). The optimal alignment is determined as the best score using the GAP program in the GCG software package with a Blossum 62 scoring matrix with a gap penalty of 12, a gap extend penalty of 4, and a frame shift gap penalty of 5. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences.

“Sequence similarity” between polynucleotides can be determined by hybridization of polynucleotides under conditions that allow formation of stable duplexes between homologous regions, followed by digestion with single-stranded-specific nuclease(s), and size determination of the digested fragments.

“Subject,” as used herein, may mean either a human or non-human animal. The term includes, but is not limited to, mammals (e.g., humans, other primates, pigs, rodents (e.g., mice and rats or hamsters), rabbits, guinea pigs, cows, horses, cats, dogs, sheep, and goats). In one embodiment, the subject is a human. In some embodiments, the subject is a mouse.

The term “therapeutically effective amount” or “effective amount” means the total amount of each active component of the composition or method that is sufficient to show meaningful benefits, i.e, a decrease in the subject's blood glucose levels, an increase in insulin levels, or an improved engraftment of the insulin producing cells in the subject.

“Treatment” of an individual (e.g. a mammal, such as a human) or a cell is any type of intervention used in an attempt to alter the natural course of the individual or cell. Treatment includes, but is not limited to, administration of a therapeutic composition, and may be performed either prophylactically or subsequent to the initiation of a pathologic event or contact with an etiologic agent. Treatment includes any desirable effect on the symptoms or pathology of a disease or condition, e.g., diabetes, and may include, for example, minimal changes or improvements in one or more measurable effects of the disease or condition being treated. Also included are “prophylactic” treatments, which can be directed to reducing the rate of progression of the disease or condition being treated, delaying the onset of that disease or condition, or reducing the severity of its onset. “Treatment” or “prophylaxis” does not necessarily indicate complete eradication, cure, or prevention of the disease or condition, or associated symptoms thereof.

“Expansion” refers to the propagation of cells without differentiation, including the proliferation of any cell type without significant further differentiation.

“Progenitor cells” are cells produced during differentiation of a stem cell that have some, but not all, of the characteristics of their terminally-differentiated progeny. Defined progenitor cells, such as “pancreatic progenitor cells,” are committed to a lineage, but not to a specific or terminally-differentiated cell type.

“Increase expression” of a marker (e.g., MMP-9) refers to an increase (in mRNA and/or protein) relative to the parent cell (a cell prior to a recited treatment or modification, e.g., contacting the cell with viral vectors or nucleic acids and/or treatments) on an average per cell basis (for example, if the parent cell does not express a marker and the progeny does, there is an increase in expression; or if the progeny expresses more of the marker compared to the parent cell there is also an increase in expression).

“Engraft” or “engraftment” refers to the process of cellular contact and incorporation into an existing tissue or site of interest. In one embodiment, greater than about 5%, greater than about 10%, greater than about 15%, greater than about 20%, greater than about 25%, greater than about 30%, greater than about 35%, greater than about 40%, greater than about 45%, greater than about 50%, greater than about 55%, greater than about 60%, greater than about 65%, greater than about 70%, greater than about 75%, greater than about 80%, greater than about 85%, greater than about 90%, greater than about 95% or about 100% of administered cells engraft in the pancreas or other tissues.

Persistence refers to the ability of cells to resist rejection and remain or increase in number over time (e.g., days, weeks, months, years) in vivo. Thus, by persisting, the cells can populate the pancreas or other tissues or remain in vivo, such as in barrier devices or other encapsulated forms.

The term “isolated” refers to a cell or cells which are not associated with one or more cells or one or more cellular components that are associated with the cell or cells in vivo. An “enriched population” refers to a relative increase in numbers of the cell of interest relative to one or more other cell types in vivo or in primary culture.

“Co-administer” can include sequential, simultaneous and/or separate administration of two or more agents.

In one embodiment, the invention provides a cellular therapeutic composition for treating diabetes in a subject comprising:
i) a therapeutically effective amount of insulin producing cells; and
ii) a therapeutically effective amount of modified mesenchymal stem cells, wherein the mesenchymal stem cells have been modified to increase expression of matrix metalloproteinase-9 (MMP-9) or a fragment or variant thereof.

In another embodiment, the invention provides a method of treating or preventing diabetes in a subject, comprising administering to the subject an effective amount of a cellular therapeutic composition comprising:
i) a therapeutically effective amount of insulin producing cells; and
ii) a therapeutically effective amount of modified mesenchymal stem cells, wherein the mesenchymal stem cells have been modified to increase expression of matrix metalloproteinase-9 (MMP-9) or a fragment or variant thereof.

The diabetes to be treated is not necessarily limiting. Generally, diabetes mellitus can be subdivided into two distinct types: Type 1 diabetes and Type 2 diabetes. Type 1 diabetes is characterized by little or no circulating insulin and it most commonly appears in childhood or early adolescence. It is caused by the destruction of the insulin-producing beta cells of the pancreatic islets. To survive, people with Type 1 diabetes must take multiple insulin injections daily and test their blood sugar multiple times per day. However, the multiple daily injections of insulin do not adequately mimic the body's minute-to-minute production of insulin and precise control of glucose metabolism. Blood sugar levels are usually higher than normal, causing complications that include blindness, renal failure, non-healing peripheral vascular ulcers, the premature development of heart disease or stroke, gangrene and amputation, nerve damage, impotence and it decreases the sufferer's overall life expectancy by one to two decades.

Type 2 diabetes usually appears in middle age or later and particularly affects those who are overweight. In Type 2 diabetes, the body's cells that normally require insulin lose their sensitivity and fail to respond to insulin normally. This insulin resistance may be overcome for many years by extra insulin production by the pancreatic beta cells. Eventually, however, the beta cells are gradually exhausted because they have to produce large amounts of excess insulin due to the elevated blood glucose levels. Ultimately, the overworked beta cells die and insulin secretion fails, bringing with it a concomitant rise in blood glucose to sufficient levels that it can only be controlled by exogenous insulin injections. High blood pressure and abnormal cholesterol levels usually accompany Type 2 diabetes. These conditions, together with high blood sugar, increase the risk of heart attack, stroke, and circulatory blockages in the legs leading to amputation.

There is a third type of diabetes in which diabetes is caused by a genetic defect, such as Maturity Onset Diabetes of the Young (MODY). MODY is due to a genetic error in the insulin-producing cells that restricts its ability to process the glucose that enters this cell via a special glucose receptor. Beta cells in patients with MODY cannot produce insulin correctly in response to glucose, resulting in hyperglycemia and require treatment that eventually also requires insulin injections.

In some embodiments, the subject has Type I diabetes. In some embodiments, the subject has Type II diabetes. In some embodiments, the subject has Maturity Onset Diabetes of the Young.

In some embodiments, the subject with diabetes has end-stage renal disease, and the methods are performed in conjunction with a kidney xenotransplant.

In some embodiments, the subject with diabetes has hypoglycemia unawareness.

The insulin producing cells are not limiting. In some embodiments, they are progenitor cells or stem cells that have the capability to produce insulin upon differentiation, which can occur following administration to the subject. In some embodiments, the insulin producing cells are differentiated from stem or progenitor cells in vitro, and then administered.

In some embodiments, the stem cells are embryonic stem cells, adult stem cells or induced pluripotent stem cells. The embryonic stem (ES) cell has unlimited self-renewal and can differentiate into all tissue types. ES cells are derived from the inner cell mass of the blastocyst or primordial germ cells from a post-implantation embryo (embryonic germ cells or EG cells). ES (and EG) cells can be identified by positive staining with antibodies to SSEA 1 (mouse) and SSEA 4 (human). At the molecular level, ES and EG cells express a number of transcription factors specific for these undifferentiated cells. These include Oct-4 and rex-1. Rex expression depends on Oct-4. Also found are LIF-R (in mouse) and the transcription factors sox-2 and rex-1. Rex-1 and sox-2 are also expressed in non-ES cells. Another hallmark of ES cells is the presence of telomerase, which provides these cells with an unlimited self-renewal potential in vitro.

Adult stem cells, such as “Multipotent adult progenitor cells” (MAPCs) are non-embryonic (non-ES), non-germ and non-embryonic germ (non-EG) cells that can differentiate into one or more ectodermal, endodermal and mesodermal cells types. MAPCs can be positive for telomerase, Oct-3A (Oct-3/4) or a combination thereof. MAPCs have the ability to regenerate all primitive germ layers (endodermal, mesodermal and ectodermal) in vitro and in vivo. In this context they are equivalent to embryonic stem cells and distinct from mesenchymal stem cells. The biological potency of MAPCs has been proven in various animal models, including mouse, rat, and xenogeneic engraftment of human stem cells in rats or NOD/SCID mice (Jiang, Y. et al. 2002). Clonal potency of this cell population has been shown. MAPCs are capable of extensive culture without loss of differentiation potential and show efficient, long term, engraftment and differentiation along multiple developmental lineages without evidence of teratoma formation.

Induced pluripotent stem cell (iPSC) technology is the process of converting an adult specialized cell, such as a skin cell, into a stem cell, a process known as dedifferentiation. Nuclear reprogramming, the process of converting one cell type into another by resetting the pattern of gene expression, can be achieved through forced expression of defined transcription factors. One example is the induced pluripotent stem cells (iPSCs), which can be prepared by transducing up to four genes (e.g., Oct4, Sox2, Klf4 and c-Myc, called OSKM hereafter) into differentiated somatic cells, such as skin fibroblasts. Other genes which can be transduced in place of or in addition to, so as to generate iPS cells, include, for example nanog and lin28.

The first mouse iPS cell line was generated in 2006, which showed ES-like characteristics in self-renewal, teratoma and chimera formation and differentiation. In 2007 human iPS cell lines were successfully established, which made the derivation of individual pluripotent stem cells possible from a small skin biopsy. These and many subsequent studies have confirmed human iPS cells are characteristically very similar to ES cells. iPS cells can self-renew and are able to maintain an undifferentiated state when grown under appropriate conditions. As pluripotent cells, they can also differentiate into any cell type, including pancreatic cells, when exposed to environment permissive for, or directing differentiation. Human iPS cell lines have been generated from normal human skin cells and diabetic donors, all of which had the potential to differentiate into insulin-producing cells.

iPS cells can also have therapeutic uses for the treatment of disease without the need for stem cells derived from an embryonic source. For example, iPSCs can be created from human patients and can be differentiated into many tissues to provide new materials for autologous transplantation, which can avoid immune rejection of the transplanted tissues. For example, pancreatic beta cells differentiated from a patient's iPSCs can be transplanted into the original patient to treat diabetes. However, before these derivatives can be used in clinic, procedures must be developed to generate large numbers of functional cells for preclinical and human trials.

In some embodiments, the insulin producing cells are pancreatic cells, such as islet cells or β-cells. In some embodiments, the pancreatic cells are isolated from the subject or a donor.

In some embodiments, the insulin producing cells are differentiated from stem cells. Pluripotent stem cells including embryonic stem (ES) cells and induced pluripotent stem (iPS) cells can be infinitely expanded in vitro and differentiated into any cell type when exposed to the appropriate signals. Previous studies have shown that human ES cells can be directed to differentiate into functional endocrine cells, and that transplantation of these pancreatic-like cells derived from human ES (hES) cells in vitro normalizes glucose levels in diabetic mice. Induced pluripotent stem cells have been generated from non-diabetic and diabetic donors, and induced to differentiate into pancreatic insulin-producing cells. Induced pluripotent stem cells have the advantage of being accessible from any individual, and thus, could provide patient-specific donor cell source for a range of diseases.

In one embodiment, the stem cells are embryonic or adult stem cells (e.g., MAPCs or MIAMI (marrow-isolated adult multilineage inducible) cells). In another embodiment, the stem cells are induced pluripotent stem (iPS) cells. In one embodiment, the stem cells are mammalian cells, such as human cells. In some embodiments, the stem cells are allogenic. In some embodiments, the stem cells are autologous.

Stem/iPS cells and pancreatic progenitor cells differentiated from stem/iPS cells are useful as a source of pancreatic cells. The maturation, proliferation and differentiation of stem/iPS cells may be effected through culturing stem/iPS cells with appropriate factors (examples of nucleotide/protein accession numbers provided) including, but not limited to, activin-A (generally two subunits of NM_002192) or other members TGFB family of cytokines (e.g., BMP-4), including, but not limited the nodal subset of the TGFB family of cytokines (activin and nodal related factors, including, but not limited to, nodal, activina and activinb), Wnt3a (or other members of the Wnt family, including, but not limited to, WNT1 (NM_005430; NM_021279; NP_005421; NP_067254), WNT2 (NM_003391; NM_023653; NP_003382; NP_076142), WNT2B (NM_004185; NM_009520; NP_004176; NP_033546), WNT3 (NM_030753; NM_009521; NP_110380; NP_033547), WNT3A (NM_033131; NM_009522; NP_149122; NP_033548), WNT4 (NM_030761; NM_009523; NP_110388; NP_033549), WNT5A (NM_003392; NM_009524; NP_003383; NP_033550), WNT5B (NM_030775; NM_009525; NP_110402; NP_033551), WNT6 (NM_006522), WNT7A (NM_004625; NM_009527; NP_004616; NP_033553), WNT7B (NM_058238; NM_009528; NP_478679; NP_033554), WNT8A, WNT8B (NM_003393; NM_011720; NP_003384; NP_035850), WNT9A (NM_003395; NM_139298; NP_003386; NP_647459), WNT9B, WNT10A (NM_025216; NM_009518; NP_079492 NP_033544), WNT10B (NM_003394; NM_011718; NP_003385; NP_035848), WNT11 (NP_004617; NP_033545; NP_004617; NP_033545), WNT16 (NM_016087; NM_053116; NP_057171 NP_444346)), an agent that inhibits sonic hedgehog activity (including, but not limited to, cyclopamine and anti-SHH antibody), HGF (Hepatocyte growth factor/scatter factor; NM_000601; NM_010427; NP_000592; NP_034557), a member of the Epidermal growth factor (EGF) family (including, but not limited to, EGF (NM_001963; NM_010113; NP_001954; NP_034243), Heparin-binding EGF-like growth factor (HB-EGF; NM_001945; NM_010415; NP_001936; NP_034545), transforming growth factor-a (TGF-a; NM_003236; NM_031199; NP_003227; NP_112476), Amphiregulin (AR; NM_001657; NM_009704; NP_001648; NP_033834), Epigen (NM_001013442), Betacellulin (BTC; NM_001729; NM_007568; NP_001720; NP_031594), neuregulin-1 (NRG1; NM_004495; XM_620642; NP_004486; XP_620642), neuregulin-2 (NRG2; XM_001129975; XP_001129975), neuregulin-3 (NRG3; NM_001165972; NM_008734; NP_001010848; NP_032760), neuregulin-4 (NRG4; NM_138573; NM_032002; NP_612640; NP_114391), any protein which contains one or more repeats of the conserved amino sequence CX7CX4-5CX10-13CXCX8GXRC, where X represents any amino acid, or a combination thereof), or other mitogenic proteins, exendin (including, but not limited to, exendin 4 (H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2; SEQ ID NO:3) and exenatide (a synthetic 39-amino acid peptide which closely resembles exendin-4 and is marketed by Amylin Pharmaceuticals and Eli Lilly and Company as Byetta™ for the treatment of diabetes; CAS number 141732-76-5)), Growth differentiation factor 11 (GDF11) also known as bone morphogenetic protein 11 (BMP-11) or other members of the bone morphogenetic protein/transforming growth factor beta (BMP/TGFbeta) superfamily, and/or betacellulin (or other members of the EGF family). These proteins can generally be used in the amount of, for example, about 0.5 to about 200 ng/ml or about 5 nM to about 30 nM.

The transforming growth factor beta (TGF-β) family is a large family of structurally related cell regulatory proteins ((LIVM)-x(2)-P-x(2)-[FY]-x(4)-C-x-G-x-C). Proteins from the TGF-beta family are generally active as a homo- or heterodimer; the two chains being linked by a disulfide bond. Members of the TGFB family of cytokines (with examples of nucleotide/protein accession numbers for these members) include, but are not limited to, AMH (NM_000479); ARTN; BMP10 (NM_014482; NM_009756; NP_055297; NP_033886); BMP15 (NM_005448; NM_009757; NP_005439; NP_005439); BMP2 (NM_001200; NM_007553; NP_001191; NP_031579); BMP3 (NM_001201; NM_173404; NP_001192; NP_775580); BMP4 (NM_001202; NM_007554; NP_001193; NP_031580); BMP5 (NM_021073; NM_007555; NP_066551; NP_031581); BMP6 (NM_001718; NM_007556; NP_001709; NP_031582); BMP7 (NM_001719; NM_007557; NP_001710; NP_031583); BMP8A (NM_181809; NM_007558; NP_861525; NP_031584); BMP8B (NM_001720; NM_001720); GDF1 (NM_001492; NM_008107; NP_001483; NP_032133); GDF10 (NM_004962; NM_145741; NP_004953; NP_665684); GDF11 (NM_005811; NM_010272; NP_005802; NM_010272); GDF15 (NM_004864; NM_011819; NP_004855; NP_035949); GDF2 (NM_016204; NM_019506; NP_057288; NP_062379); GDF3 (NM_020634; NM_008108; NP_065685; NP_032134); GDF3A; GDF5 (NM_000557; NM_008109; NP_000548; NP_032135); GDF6 (NM_001001557; NM_013526; NP_038554); GDF7 (NM_182828; NM_013527; NP_878248; NP_038555); GDF8 (NM_005259; NM_010834; NP_005250; NP_034964); GDF9 (NM_005260; NM_008110; NP_005251; NP_032136); GDNF (NM_000514; NM_010275; NP_000505; NP_034405); INHA (NM_002191; NM_010564; NP_002182; NP_034694); INHBA (NM_002192; NM_008380; NP_002183; NP_032406); INHBB (NM_002193; XM_984243; NP_002184; XP_989337); INHBC (NM_005538; NM_010565; NP_005529; NP_034695); INHBE; LEFTY1; LEFTY2; MSTN (NM_005259; NM_010834; NP_005250; NP_034964); NODAL (NM_018055; NM_013611; NP_060525; NP_038639); NRTN (NM_004558); PSPN; TGFB1 (NM_000660; NM_011577; NP_000651; NP_035707); TGFB2 (NM_003238; NM_009367; NP_003229; NP_033393); and TGFB3 (NM_003239; XM_994378; NP_003230; XP_999472).

For example, sequences for use in the invention have at least about 50% or about 60% or about 70%, about 71%, about 72%, about 73%, about 74%, about 75%, about 76%, about 77%, about 78%, or about 79%, or at least about 80%, about 81%, about 82%, about 83%, about 84%, about 85%, about 86%, about 87%, about 88%, or about 89%, or at least about 90%, about 91%, about 92%, about 93%, or about 94%, or at least about 95%, about 96%, about 97%, about 98%, or about 99% sequence identity compared to the accession numbers provided herein and/or any other such sequence available to an art worker, using one of alignment programs available in the art using standard parameters. In one embodiment, the differences in sequence are due to conservative amino acid changes. In another embodiment, the protein sequence or DNA sequence has at least 80%, at least 85%, at least 90%, at least 95% sequence identity with the sequences disclosed herein and is bioactive (e.g., retains activity).

Methods of identifying and subsequently separating differentiated cells from their undifferentiated counterparts can be carried out by methods well known in the art and described herein. Cells that have been induced to differentiate can be identified by selectively culturing cells under conditions whereby differentiated cells outnumber undifferentiated cells. Similarly, differentiated cells can be identified by morphological changes and characteristics that are not present on their undifferentiated counterparts, such as cell size, the number of cellular processes, the complexity of intracellular organelle distribution, and the production of insulin or C-peptide and the secretion of insulin or C-peptide in response to glucose.

Also contemplated are methods of identifying differentiated cells by their expression of specific cell-surface markers such as cellular receptors and transmembrane proteins. Monoclonal antibodies against these cell-surface markers can be used to identify differentiated cells. Detection of these cells can be achieved through fluorescence activated cell sorting (FACS) and enzyme-linked immunosorbent assay (ELISA). From the standpoint of transcriptional upregulation (or increase protein expression) of specific genes, differentiated cells often display levels of gene expression that are different (increased or decreased expression of mRNA or protein) from undifferentiated cells, such as insulin-1, insulin-2, glucagon, somatostatin, NeuroD1, Pdx-1, Ngn3, Nkx6.1, Nkx2.2, rfx-6, ptf1, glucokinase (glck), chromogranin, Maf, and/or glucose transporter. Reverse-transcription polymerase chain reaction (RT-PCR) can be used to monitor such changes in gene expression during differentiation. In addition, whole genome analysis using microarray technology can be used to identify differentiated cells.

Accordingly, once differentiated cells are identified, they can be separated from their undifferentiated counterparts, if necessary. The methods of identification detailed above also provide methods of separation, such as FACS, preferential cell culture methods, ELISA, magnetic beads, and combinations thereof.

In some embodiments, the insulin producing cells are capable of secreting an effective amount of insulin when administered to the subject, thereby lowering blood sugar levels in the subject and treating diabetes. The amount of insulin produced by the insulin producing cells in the subject can vary depending on several factors, including whether the subject is fasting or has recently eaten. In some embodiments, the subject has fasting insulin levels (no food intake for at least 8 hours) of between about 2 to 20 uU/mL (microunits per milliliter). In some embodiments, the subject has postprandial (after eating) insulin levels of up to about 50-60 U/mL within about 30 to 60 minutes after a meal.

The mesenchymal stem cells that can be modified are not necessarily limiting. Mesenchymal stem cells (MSCs) are multipotent stromal cells capable of differentiating into a variety of cell types, including osteoblasts, chondrocytes, and adipocytes. MSCs can be isolated from various tissues, such as bone marrow (aspirates), adipose tissue (liposuction aspirates), umbilical cord blood, placenta, dental pulp, amniotic fluid, and peripheral blood.

In some embodiments, the mesenchymal stem cells are allogenic. In some embodiments, the mesenchymal stem cells are autologous. In some embodiments, the mesenchymal stem cells are derived from bone marrow.

The mesenchymal stem cells have been modified to increase expression of MMP-9 or a fragment or variant thereof. The methods of modifying the MSC are not limiting.

In some embodiments, MMP-9 expression is increased by reducing expression of SMAD4. In some embodiments, the SMAD4 expression is reduced by administering to the mesenchymal stem cells a CRISPR-Cas9 system using guide RNAs (gRNA1-AACTCTGTACAAAGACCGCG (SEQ ID NO:4); gRNA2-TTCTTCCTAAGGTTGCACAT) (SEQ ID NO:5). In some embodiments, the gRNAs can be cloned into a viral vector, such as a lentiviral vector, e.g., pLenti-CRISPR-V2-SMAD4. In some embodiments, the SMAD4 expression is reduced by administering to the mesenchymal stem cells a nucleic acid. In some embodiments, the nucleic acid is a siRNA. The siRNA is not particularly limiting. Various siRNA can be obtained, for example, from commercial sources. See, e.g., siRNA ID No: s8405 (ThermoFisher Scientific).

In some embodiments, the mesenchymal stem cells have been modified to increase expression of MMP-9 by administering to the mesenchymal stem cells a CRISPRa/dCas9 and sgRNA (sequence; GCAGTGGAGAGAGGAGGAGG) (SEQ ID NO:6) that binds to an upstream promoter region of MMP-9. In some embodiments, the gRNAs can be cloned in construct MMP9 SAM guide RNA 1pLentisgRNAMS2zeo and targeted to MSCs.

In some embodiments, the mesenchymal stem cells have been modified to increase expression of MMP-9 or a fragment or variant thereof, by administering to the mesenchymal stem cells a nucleic acid that encodes MMP-9 or a fragment or variant thereof.

In some embodiments, the MMP-9 protein has the amino acid sequence found in NCBI accession number AY889934 and has the amino acid sequence comprising SEQ ID NO: 1. In some embodiments, MMP-9 has a nucleotide sequence comprising SEQ ID NO: 2.

TABLE 1
MMP-9 amino acid and nucleotide sequences

| SEQ ID | |
|---|---|
| NO: | Sequence |
| 1 | MSLWQPLVLVLLVLGCCFAAPRQRQSTLVLFPGDLRTNLTDRQLAEEYLYRYGYT |
| | RVAEMRGESKSLGPALLLLQKQLSLPETGELDSATLKAMRTPRCGVPDLGRFQTF |
| | EGDLKWHHHNITYWIQNYSEDLPRAVIDDAFARAFALWSAVTPLTFTRVYSRDAD |
| | IVIQFGVAEHGDGYPFDGKDGLLAHAFPPGPGIQGDAHFDDDELWSLGKGVVVPT |
| | RFGNADGAACHFPFIFEGRSYSACTTDGRSDGLPWCSTTANYDTDDRFGFCPSER |
| | LYTRDGNADGKPCQFPFIFQGQSYSACTTDGRSDGYRWCATTANYDRDKLFGFCP |
| | TRADSTVMGGNSAGELCVFPFTFLGKEYSTCTSEGRGDGRLWCATTSNFDSDKKW |
| | GFCPDQGYSLFLVAAHEFGHALGLDHSSVPEALMYPMYRFTEGPPLHKDDVNGIR |
| | HLYGPRPEPEPRPPTTTTPQPTAPPTVCPTGPPTVHPSERPTAGPTGPPSAGPTG |
| | PPTAGPSTATTVPLSPVDDACNVNIFDAIAEIGNQLYLFKDGKYWRFSEGRGSRP |
| | QGPFLIADKWPALPRKLDSVFEEPLSKKLFFFSGRQVWVYTGASVLGPRRLDKLG |
| | LGADVAQVTGALRSGRGKMLLFSGRRLWRFDVKAQMVDPRSASEVDRMFPGVPLD |
| | THDVFQYREKAYFCQDRFYWRVSSRSELNQVDQVGYVTYDILQCPED |
| 2 | atgagcctct ggcagcccct ggtcctggtg ctcctggtgc tgggctgctg |
| | ctttgctgcccccagacagc gccagtccac ccttgtgctc ttccctggag acctgagaac |
| | caatctcaccgacaggcagc tggcagagga atacctgtac cgctatggtt acactcgggt |
| | ggcagagatgcgtggagagt cgaaatctct ggggcctgcg ctgctgcttc tccagaagca |
| | actgtccctgcccgagaccg gtgagctgga tagcgccacg ctgaaggcca tgcgaacccc |
| | acggtgcggggtcccagacc tgggcagatt ccaaaccttt gagggcgacc tcaagtggca |
| | ccaccacaacatcacctatt ggatccaaaa ctactcggaa gacttgccgc gggcggtgat |
| | tgacgacgcctttgcccgcg ccttcgcact gtggagcgcg gtgacgccgc tcaccttcac |
| | tcgcgtgtacagccgggacg cagacatcgt catccagttt ggtgtcgcgg agcacggaga |
| | cgggtatcccttcgacggga aggacgggct cctggcacac gcctttcctc ctggccccgg |
| | cattcagggagacgcccatt tcgacgatga cgagttgtgg tccctgggca agggcgtcgt |
| | ggttccaactcggtttggaa acgcagatgg cgcggcctgc cacttcccct tcatcttcga |
| | gggccgctcctactctgcct gcaccaccga cggtcgctcc gacggcttgc cctggtgcag |
| | taccacggccaactacgaca ccgacgaccg gtttggcttc tgccccagcg agagactcta |
| | cacccgggacggcaatgctg atgggaaacc ctgccagttt ccattcatct tccaaggcca |
| | atcctactccgcctgcacca cggacggtcg ctccgacggc taccgctggt gcgccaccac |
| | cgccaactacgaccgggaca agctcttcgg cttctgcccg acccgagctg actcgacggt |
| | gatggggggcaactcggcgg gggagctgtg cgtcttcccc ttcactttcc tgggtaagga |
| | gtactcgacctgtaccagcg agggccgcgg agatgggcgc ctctggtgcg ctaccacctc |
| | gaactttgacagcgacaaga agtggggctt ctgcccggac caaggataca gtttgttcct |
| | cgtggcggcgcatgagttcg gccacgcgct gggcttagat cattcctcag tgccggaggc |
| | gctcatgtaccctatgtacc gcttcactga ggggcccccc ttgcataagg acgacgtgaa |
| | tggcatccggcacctctatg gtcctcgccc tgaacctgag ccacggcctc caaccaccac |
| | cacaccgcagcccacggctc ccccgacggt ctgccccacc ggacccccca ctgtccaccc |
| | ctcagagcgacccacagctg gccccacagg tcccccctca gctggcccca caggtccccc |
| | cactgctggcccttctacgg ccactactgt gcctttgagt ccggtggacg atgcctgcaa |
| | cgtgaacatcttcgacgcca tcgcggagat tgggaaccag ctgtatttgt tcaaggatgg |
| | gaagtactggcgattctctg agggcagggg gagccggccg cagggcccct tccttatcgc |
| | cgacaagtggcccgcgctgc cccgcaagct ggactcggtc tttgaggagc cgctctccaa |
| | gaagcttttcttcttctctg ggcgccaggt gtgggtgtac acaggcgcgt cggtgctggg |
| | cccgaggcgtctggacaagc tgggcctggg agccgacgtg gcccaggtga ccggggccct |
| | ccggagtggcagggggaaga tgctgctgtt cagcgggcgg cgcctctgga ggttcgacgt |
| | gaaggcgcagatggtggatc cccggagcgc cagcgaggtg gaccggatgt tccccggggt |
| | gcctttggacacgcacgacg tcttccagta ccgagagaaa gcctatttct gccaggaccg |
| | cttctactggcgcgtgagtt cccggagtga gttgaaccag gtggaccaag tgggctacgt |
| | gacctatgacatcctgcagt gccctgagga ctag |

In some embodiments, the nucleic acid encoding MMP-9 or a fragment or variant thereof is delivered to the MSC using a vector.

The nucleic acid vectors used in the compositions and methods described herein include polynucleotide sequences that encode MMP-9 or fragments or variants thereof, such as polynucleotide sequences that encode a protein having at least 85% sequence identity (e.g., 85%, 90%, 95%, 96%, 97%, 98%, 99%, or more, sequence identity) to the amino acid sequence of SEQ ID NO:1.

According to the methods described herein, MSCs can be administered a composition comprising a nucleic acid vector of a polynucleotide sequence encoding the amino acid sequence of SEQ ID NO:1, or a polynucleotide sequence encoding an amino acid sequence having at least 85% sequence identity (e.g., 85%, 90%, 95%, 96%, 97%, 98%, 99%, or more, sequence identity) to the amino acid sequence of SEQ ID NO:1, or a polynucleotide sequence encoding an amino acid sequence that contains one or more conservative amino acid substitutions relative to SEQ ID NO:1 (e.g., 1, 2, 3, 4, 5, 6, 7, 8, 9, 10, 11, 12, 13, 14, 15, 16, 17, 18, 19, 20 or more conservative amino acid substitutions), provided that the variant encoded retains the therapeutic function of MMP-9. In some embodiments, the variant comprises an amino acid sequence that is at least 90% identical to SEQ ID NO:1.

In some embodiments, no more than 10% of the amino acids of MMP-9 may be replaced with conservative amino acid substitutions. In some embodiments, the MMP-9 may be encoded by a polynucleotide having the sequence of SEQ ID NO:2. Variants of the polynucleotide sequence can also be used, for example, due to degeneracy of codon usage.

The organismal source of the nucleic acid sequence encoding MMP-9 is not limiting. In some embodiments, MMP-9 can be a homolog of human MMP-9 from another mammalian species (e.g., mouse, rat, cow, horse, goat, sheep, donkey, cat, dog, rabbit, guinea pig, or other mammal). In some embodiments, the nucleic acid sequence is derived from a mammal. In some embodiments, the nucleic acid sequence is of human origin.

The nucleic acid molecule encoding MMP-9 or a fragment or variant thereof can be produced using recombinant DNA technology (e.g., polymerase chain reaction (PCR) amplification, cloning) or chemical synthesis. Nucleic acids that encode MMP-9 include natural nucleic acid molecules and homologues thereof, including, but not limited to, natural allelic variants and modified nucleic acid molecules in which nucleotides have been inserted, deleted, substituted, and/or inverted in such a manner that such modifications provide the desired effect (e.g., production of MMP-9 protein in cells or other expression systems).

In some embodiments, the coding sequence of MMP-9 is encoded by SEQ ID NO: 2. The nucleic acid encoding MMP-9 in accordance with the invention may contain a variety of different bases compared to the wild-type sequence and yet still encode a corresponding polypeptide that exhibits the biological activity of the native MMP-9 polypeptide.

In some embodiments, a particular nucleotide sequence encoding MMP-9 polypeptide may be identical over its entire length to the coding sequence in SEQ ID NO:2. In some embodiments, a particular nucleotide sequence encoding MMP-9 polypeptide may be an alternate form of SEQ ID NO:2 due to degeneracy in the genetic code or variation in codon usage encoding the polypeptide of SEQ ID NO:1.

In some embodiments, the nucleic acid sequence of MMP-9 can contain a nucleotide sequence that is highly identical, at least 60% identical, with a nucleotide sequence encoding MMP-9 polypeptide. In some embodiments, the nucleic acid sequence of MMP-9 comprises a nucleotide sequence that is at least 60%, 65%, 70%, 75%, 80%, 85%, 90%, 91%, 92%, 93%, 94%, 95%, 96%, 97%, 98% 99% or 100% identical with the encoding nucleotide sequence set forth in SEQ ID NO:2.

When a polynucleotide of the invention is used for the production of MMP-9 polypeptide, the polynucleotide may include the coding sequence for the full-length polypeptide or a fragment thereof, by itself; the coding sequence for the full-length polypeptide or fragment in reading frame with other coding sequences, such as those encoding a leader or secretory sequence, a pre-, or pro or prepro-protein sequence, or other fusion peptide portions. The polynucleotide may also contain non-coding 5′ and 3′ sequences, such as transcribed, non-translated sequences, splicing and polyadenylation signals, ribosome binding sites and sequences that stabilize mRNA.

In some embodiments, the nucleotide sequence used in vector encoding the MMP-9 polypeptide or a biologically active fragment or derivative thereof includes nucleic acid molecules comprising a polynucleotide having a nucleotide sequence at least 90%, 91%, 92%, 93%, 94%, 95%, 96%, 97%, 98% 99% or 100% identical to (a) a nucleotide sequence encoding MMP-9 having the amino acid sequence in SEQ ID NO:1; or (b) a nucleotide sequence complementary to the nucleotide sequences in (a).

Conventional means utilizing known computer programs such as the BestFit program (Wisconsin Sequence Analysis Package, Version 10 for Unix, Genetics Computer Group, University Research Park, 575 Science Drive, Madison, Wis. 53711) may be utilized to determine if a particular nucleic acid or polypeptide molecule is at least 90%, 91%, 92%, 93%, 94%, 95%, 96%, 97%, 98% or 99% identical to SEQ ID NO: 1 or 2, for example.

In some embodiments, the nucleotide sequence used encoding MMP-9 or a fragment or variant thereof encodes an amino acid sequence of MMP-9 of SEQ ID NO:1, in which 1, 2, 3, 4, 5, 6, 7, 8, 9, 10 or more amino acid residues are substituted, deleted or added, in any combination.

In some embodiments, the nucleotide sequence are at least 90% identical over their entire length to a polynucleotide encoding the corresponding portion of MMP-9, for which the amino acid sequence is set out in SEQ ID NO:1, and polynucleotides which are complementary to such polynucleotides. In some embodiments, the polynucleotides are at least 91%, 92%, 93%, 94%, 95%, 96%, 97%, 98%, or at least 99% identical.

In some embodiments, the nucleic acid molecule encodes a variant of MMP-9 protein which is a biologically active fragment. In some embodiments, the biologically active fragment can be at least about 400, 500, 550, 600, 650, 660, 670, 680, 690, 700 or more amino acids in length.

Stable expression of MMP-9 or a fragment or variant thereof in a MSC can be achieved by integration of the polynucleotides containing the MMP-9 or variant thereof into the nuclear genome of the cell. A variety of vectors for the delivery and integration of polynucleotides encoding exogenous protein into the nuclear DNA of a mammalian cell have been developed. In some embodiments, expression vectors for use in the compositions and methods described herein contain a polynucleotide sequence that encodes a portion of MMP-9 or variant thereof, as well as, e.g., additional sequence elements used for the expression of these agents and/or the integration of these polynucleotide sequences into the genome of a mammalian cell. Certain vectors that can be used for the expression of MMP-9 include plasmids that contain regulatory sequences, such as promoter and enhancer regions, which direct gene transcription. Other useful vectors for expression of MMP-9 contain polynucleotide sequences that enhance the rate of translation of these genes or improve the stability or nuclear export of the mRNA that results from gene transcription. These sequence elements include, e.g., 5′ and 3′ untranslated regions and a polyadenylation signal site in order to direct efficient transcription of the gene carried on the expression vector. The expression vectors suitable for use with the compositions and methods described herein may also contain a polynucleotide encoding a marker for selection of cells that contain such a vector. Examples of a suitable marker include genes that encode resistance to antibiotics, such as ampicillin, chloramphenicol, kanamycin, or nourseothricin.

In some embodiments, the compositions and methods described herein increase the expression of MMP-9 or fragments of variants thereof by administering a nucleic acid vector that contains a polynucleotide encoding MMP-9 or a fragment or variant thereof.

Vectors can be introduced into a cell by a variety of methods, including transformation, transfection, transduction, direct uptake, projectile bombardment, and by encapsulation of the vector in a liposome. Examples of suitable methods of transfecting or transforming cells include calcium phosphate precipitation, electroporation, microinjection, infection, lipofection and direct uptake. Such methods are described in more detail, for example, in Green, et al., Molecular Cloning: A Laboratory Manual, Fourth Edition (Cold Spring Harbor University Press, New York 2014); and Ausubel, et al., Current Protocols in Molecular Biology (John Wiley & Sons, New York 2015), the disclosures of each of which are incorporated herein by reference.

In some embodiments, MMP-9 or fragments of variants thereof can also be introduced into a mammalian cell by targeting vectors encoding MMP-9 or fragments of variants thereof to cell membrane phospholipids. For example, vectors can be targeted to the phospholipids on the extracellular surface of the cell membrane by linking the vector molecule to a VSV-G protein, a viral protein with affinity for all cell membrane phospholipids. Such a construct can be produced using methods well known to those of skill in the field.

Recognition and binding of the polynucleotide encoding MMP-9 or fragments of variants thereof by mammalian RNA polymerase is important for gene expression. As such, one may include sequence elements within the polynucleotide that exhibit a high affinity for transcription factors that recruit RNA polymerase and promote the assembly of the transcription complex at the transcription initiation site.

Such sequence elements include, e.g., a mammalian promoter, the sequence of which can be recognized and bound by specific transcription initiation factors and ultimately RNA polymerase.

Polynucleotides suitable for use in the compositions and methods described herein also include those that encode an MMP-9 protein downstream of a mammalian promoter (e.g., a polynucleotide that encodes an N-terminal portion of an MMP-9 downstream of a mammalian promoter). Promoters that are useful for the expression of an MMP-9 protein in mammalian cells include ubiquitous promoters and cochlear hair cell-specific promoters. Ubiquitous promoters include the CAG promoter, or the cytomegalovirus (CMV) promoter. Cell type and tissue specific promoters can also be utilized.

Alternatively, promoters derived from viral genomes can also be used for the stable expression of these agents in mammalian cells. Examples of functional viral promoters that can be used to promote mammalian expression of these agents include adenovirus late promoter, vaccinia virus 7.5K promoter, SV40 promoter, tk promoter of HSV, mouse mammary tumor virus (MMTV) promoter, LTR promoter of HIV, promoter of Moloney virus, Epstein barr virus (EBV) promoter, and the Rous sarcoma virus (RSV) promoter.

In some embodiments, the nucleic acid is delivered to cells by a viral vector. In some embodiments, the virus is a DNA virus (e.g., dsDNA or ssDNA virus). In other embodiments, the virus is an RNA virus (e.g., an ssRNA virus). Exemplary viral vectors/viruses include retroviruses, lentiviruses, adenovirus, adeno-associated virus (AAV), vaccinia viruses, poxviruses, and herpes simplex viruses.

In some embodiments, the virus infects dividing cells. In other embodiments, the virus infects non-dividing cells. In some embodiments, the virus infects both dividing and non-dividing cells. In some embodiments, the virus can integrate into the host genome. In some embodiments, the virus is engineered to have reduced immunity, e.g., in humans. In some embodiments, the virus is replication-competent. In other embodiments, the virus is replication-defective, e.g., having one or more coding regions for the genes necessary for additional rounds of virion replication and/or packaging replaced with other genes or deleted. In some embodiments, the virus causes transient expression of the MMP-9 protein, fragment or variant molecule. In other embodiments, the virus causes long-lasting, e.g., at least 1 week, 2 weeks, 1 month, 2 months, 3 months, 6 months, 9 months, 1 year, 2 years, or permanent expression, of the MMP-9 protein, fragment or variant molecule. The packaging capacity of the viruses may vary, e.g., from at least about 4 kb to at least about 30 kb, e.g., at least about 5 kb, 10 kb, 15 kb, 20 kb, 25 kb, 30 kb, 35 kb, 40 kb, 45 kb, or 50 kb.

In some embodiments, the nucleic acid is delivered by a recombinant retrovirus. In some embodiments, the retrovirus (e.g., Moloney murine leukemia virus) comprises a reverse transcriptase, e.g., that allows integration into the host genome. In some embodiments, the retrovirus is replication-competent. In other embodiments, the retrovirus is replication-defective, e.g., having one of more coding regions for the genes necessary for additional rounds of virion replication and packaging replaced with other genes, or deleted.

In some embodiments, the nucleic acid is delivered by a recombinant lentivirus. In some embodiments, the lentivirus is replication-defective and does not comprise one or more genes required for viral replication.

In some embodiments, the nucleic acid is delivered by a recombinant adenovirus. In some embodiments, the adenovirus is engineered to have reduced immunity in human. In some embodiments, the nucleic acid is delivered by a recombinant AAV. In some embodiments, the AAV can incorporate its genome into that of a host cell. In some embodiments, the AAV is a self-complementary adeno-associated virus (scAAV), e.g., a scAAV that packages both strands which anneal together to form double stranded DNA. AAV serotypes that can be used in the methods of the invention include, e.g., AAVI, AAV2, modified AAV2 (e.g., modifications at Y444F, Y500F, Y730F and/or S662V), AAV3, modified AAV3 (e.g., modifications at Y705F, Y731 F and/or T492V), AAV4, AAV5, AAV6, modified AAV6 (e.g., modifications at S663V and/or T492V), AAV8, AAV 8.2, AAV9, AAV rh 10, and pseudotyped AAV, such as AAV2/8, AAV2/5 and AAV2/6 can also be used in the disclosed methods.

In some embodiments, the nucleic acid is delivered by a hybrid virus, e.g., a hybrid of one or more of the viruses described herein.

In some embodiments, a packaging cell can be used to form a virus particle that is capable of infecting a host or target cell. Such a cell can include a 293 cell, which can package adenovirus. A viral vector used in gene therapy is usually generated by a producer cell line that packages a nucleic acid vector into a viral particle. The vector typically contains the minimal viral sequences required for packaging and subsequent integration into a host or target cell (if applicable), with other viral sequences being replaced by an expression cassette encoding the protein to be expressed. For example, an AAV vector typically only possesses inverted terminal repeat (ITR) sequences from the AAV genome which are required for packaging and gene expression in the host or target cell. The missing viral functions can be supplied in trans by the packaging cell line. The viral nucleic acid can be packaged in a cell line, which contains a helper plasmid encoding the other AAV genes, namely rep and cap, but lacking ITR sequences. The cell line can also be infected with adenovirus as a helper. The helper virus can promote replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In some embodiments, the nucleic acid is delivered by a non-vector based method (e.g., using naked DNA or DNA complexes). For example, the nucleic acid can be delivered by organically modified silica or silicate (Ormosil), electroporation, gene gun, sonoporation, magnetofection, lipid-mediated transfection, dendrimers, inorganic nanoparticles, calcium phosphates, or a combination thereof.

In some embodiments, the nucleic acid is delivered by a combination of a vector and a non-vector based method. For example, a virosome comprises a liposome combined with an inactivated virus (e.g., HIV or influenza virus), which can result in more efficient gene transfer than either a viral or a liposomal method alone.

In some embodiments, nucleic acid can be delivered into cells by microinjection, electroporation, lipid-mediated transfection, peptide-mediated delivery, or a combination thereof. Delivery can be accompanied by DNA encoding a gRNA or by a gRNA.

The MMP-9 constructs described herein may be delivered or introduced into a target cell by any suitable means, including, for example, by injection of mRNA or accordingly nucleic acid, for example, a CDNA, CRNA, or IRNA. See, Hamrnerschmidt et al. (1999) Methods Cell Biol. 59:87-115.

In some embodiments, the insulin producing cells and modified mesenchymal stem cells of the therapeutic composition are incorporated into a biopolymer or synthetic polymer. Examples of biopolymer include, but are not limited to, fibronectin, fibrin, fibrinogen, thrombin, collagen and proteoglycans. In some embodiments, the cell therapeutic composition further comprises one or more cytokines, differentiation factors, angiogenesis factors or anti-apoptosis factors. In some embodiments, the cells are in suspension. In some embodiments, the cells are within a three-dimensional gel with cells entrapped within the interstices of the cell biopolymer admixture. Cytokines, differentiation factors, angiogenesis factors, anti-apoptosis factors or a combination thereof can be included within the gel.

The cells can also be co-encapsulated with a capsule that is permeable to nutrients and oxygen while allowing appropriate cellular products (for example, insulin in the case of islet cells) to be released into the bloodstream or to adjacent tissues. In one embodiment, the capsular material is restrictive enough to exclude immune cells and antibodies that could reject and destroy the implant. Such encapsulation can be achieved using polymers. Such polymeric encapsulation systems include, but are not limited to, alginate (e.g., alginate bead), polysaccharide hydrogels, chitosan, calcium or barium alginate, a layered matrix of alginate and polylysine, a photopolymerizable poly (ethylene glycol) (PEG) polymer (Novocell, Inc.), a polyanionic material termed Biodritin (U.S. Pat. No. 6,281,341), polyacrylates, a photopolymerizable poly (ethylene glycol) polymer, and polymers such as hydroxyethyl methacrylate methyl methacrylate. Another approach to encapsulate cells involves the use of photolithography techniques adapted from the semiconductor industry to encapsulate living cells in silicon capsules that have pores only a few nanometers wide. Other polymers that can be used include suitable immune-compatible polycations, including but not limited to, poly-1-lysine (PLL) polycation or poly-1-ornithine or poly (methylene-co-guanidine) hydrochloride.

In some embodiments, cells can be encapsulated with biocompatible semipermeable membranes to surround encapsulated cells, sometimes within a capillary device, to create a miniature artificial organ, such as one that would include functional pancreas or liver cells (e.g., a liver or pancreatic artificial device). This is often called macroencapsulation. The membrane lets glucose, oxygen, and insulin pass in and out of the blood stream, and preferably keeps out the antibodies and T cells of the immune system, which may destroy the cells (e.g., islets). Such membranes can be used in a perfusion device, a capsule that is grafted to an artery where it makes direct contact with the body's circulating blood; in this way, the device can draw nutrients from the blood and release insulin to circulate throughout the body.

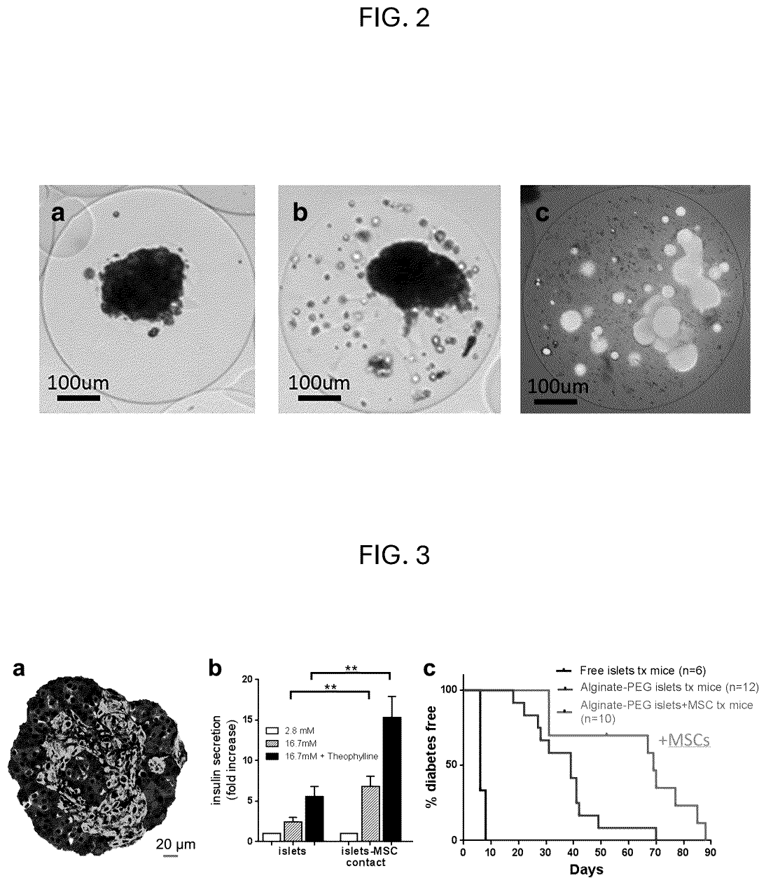

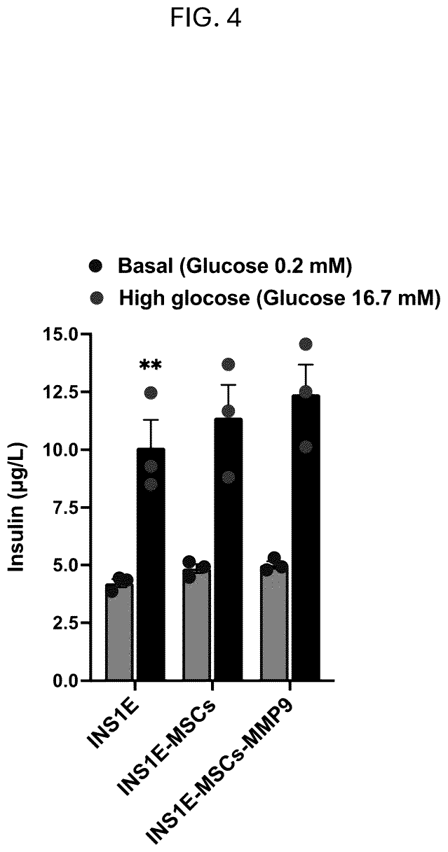

In some embodiments, the insulin producing cells and modified mesenchymal stem cells are encapsulated by a semi-permeable scaffold comprising alginate. In some embodiments, the semi-permeable scaffold comprises calcium and alginate. In some embodiments, the insulin producing cells and modified mesenchymal stem cells are co-encapsulated in calcium-alginate microbeads of about 550 μm diameter. Both MSCs and islets are co-encapsulated in Calcium-alginate microbeads of 550 μm diameter (FIG. 2) using a clinical-grade BioRep encapsulator. Methods for encapsulation are described, for example, in Montanari et al., Beneficial Effects of Human Mesenchymal Stromal Cells on Porcine Hepatocyte Viability and Albumin Secretion. J Immunol Res, 2018. 2018: p. 1078547.

The pancreatic progenitor or insulin producing cells and modified MSC can be used to repopulate a pancreas or other tissue by either direct introduction into the area of damage or by systemic administration, which allows the cells to home to an area of damage. Accordingly, the invention provides methods of treating a subject in need of pancreatic cells comprising administering to a subject an effective amount of the cell therapeutic composition herein.

For the purposes described herein, either autologous, allogeneic or xenogeneic cells can be administered to a patient, either in undifferentiated, terminally differentiated or in a partially differentiated form, by direct introduction to a site of interest, e.g., on or around the surface of an acceptable matrix, or systemically, in combination with a pharmaceutically acceptable carrier so as to repair, replace or promote the growth of existing and/or new pancreatic cells.

The cell therapeutic composition can be administered to a subject by a variety of methods available to the art, including but not limited to localized injection, catheter administration, systemic injection, intraperitoneal injection, parenteral administration, intra-arterial injection, intravenous injection, transvascular injection, intramuscular injection, subcutaneous placement/injection, surgical injection into a tissue of interest (e.g., injection into the pancreas) or via direct application to tissue surfaces (e.g., during surgery or on a wound).

In some embodiments, the cellular therapeutic composition is administered to the subject by infusion through the subject's portal vein. In some embodiments, the cellular therapeutic composition is administered to the subject intraperitoneally. In some embodiments, engraftment of the insulin producing cells is enhanced by the modified MSC compared with engraftment in the absence of the modified MSC following administration. In some embodiments, the insulin producing cells secrete an effective amount of insulin in response to the subject's blood glucose levels, thereby lowering blood glucose levels in the subject and treating diabetes.

In some embodiments, the cell therapeutic composition can be administered either peripherally or locally through the circulatory system. “Homing” of cells would concentrate the implanted cells in an environment favorable to their growth and function. Pre-treatment of a patient with cytokine(s) to promote homing is another alternative contemplated in the methods of the present invention. Certain cytokines (e.g., cellular factors that induce or enhance cellular movement, such as homing of stem cells, progenitor cells or differentiated cells) can enhance the migration of cell derived pancreatic progenitors or their progeny. Cytokines include, but are not limited to, stromal cell derived factor-1 (SDF-1), stem cell factor (SCF), angiopoietin-1, placenta-derived growth factor (PIGF) and granulocyte-colony stimulating factor (G-CSF). Cytokines also include any which promote the expression of endothelial adhesion molecules, such as ICAMs, VCAMs and others, which facilitate the homing process.

In some embodiments, viability of newly forming tissues can be enhanced by angiogenesis. Factors promoting angiogenesis include, but are not limited to, VEGF, aFGF, angiogenin, angiotensin-1 and -2, betacellulin, bFGF, Factor X and Xa, HB-EGF, PDGF, angiomodulin, angiotropin, angiopoetin-1, prostaglandin E1 and E2, steroids, heparin, 1-butyryl-glycerol and nicotinic amide.

In some embodiments, factors that decrease apoptosis can also promote the formation of new tissue, such as pancreatic tissues. Factors that decrease apoptosis include but are not limited to β-blockers, angiotensin-converting enzyme inhibitors (ACE inhibitors), AKT, HIF, carvedilol, angiotensin II type 1 receptor antagonists, caspase inhibitors, cariporide and eniporide.

Exogenous factors (e.g., cytokines, differentiation factors (e.g., cellular factors, such as growth factors or angiogenic factors that induce lineage commitment), angiogenesis factors and anti-apoptosis factors) can be administered prior to, after or concomitantly with the cell therapeutic composition.

In some embodiments, the cells can also be administered via a device or scaffolding substance (that may or may not be a polymer) to contain the cells (e.g., the cells can be placed in the device prior to implantation). In one embodiment, this device/substance is retrievable. In another embodiment, it is absorbable. In another embodiment, a site may be created surgically to contain the cells. In one embodiment, the insulin producing cells and modified MSC are transplanted with additional cell types, including, but not limited to, endothelial cells.

The quantity of cells to be administered will vary for the subject being treated. In some embodiments, between about 10⁴ to about 10⁸ or about 10⁵ to about 10⁷ cells are administered. In some embodiments, about 50 to about 500 μg/kg per day of a cytokine is administered to a subject. However, the precise determination of what would be considered an effective dose may be based on factors individual to each patient, including their size, age, disease or injury, amount of damage, amount of time since the damage occurred and factors associated with the mode of delivery (direct injection—lower doses, intravenous—higher doses).

When administering a cell therapeutic composition of the present invention, it can generally be formulated in a unit dosage injectable form (solution, suspension, emulsion). The pharmaceutical formulations suitable for injection include sterile aqueous solutions and dispersions. The carrier can be a solvent or dispersing medium containing, for example, water, saline, phosphate buffered saline, polyol (for example, glycerol, propylene glycol, liquid polyethylene glycol, and the like) and suitable mixtures thereof.

Additionally, various additives which enhance the stability, sterility, and isotonicity of the compositions, including antimicrobial preservatives, antioxidants, chelating agents and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used should be compatible with the cells.

Sterile injectable solutions can be prepared by incorporating the cells utilized in practicing the present invention in the required amount of the appropriate solvent with various amounts of the other ingredients, as desired.

In one embodiment, the cells described herein can be administered initially, and thereafter maintained by further administration of cells. For instance, the cells can be administered by one method of injection, and thereafter further administered by a different or the same type of method.

In some embodiments, compositions can be provided as liquid preparations, e.g., isotonic aqueous solutions, suspensions, emulsions or viscous compositions, which may be buffered to a selected pH. Liquid preparations are normally easier to prepare than gels, other viscous compositions and solid compositions. In some embodiments, viscous compositions can be formulated within the appropriate viscosity range to provide longer contact periods with specific tissues.

The choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

In some embodiments, solutions, suspensions and gels normally contain a major amount of water (preferably purified, sterilized water) in addition to the cells. Minor amounts of other ingredients such as pH adjusters (e.g., a base such as NaOH), emulsifiers or dispersing agents, buffering agents, preservatives, wetting agents and jelling agents (e.g., methylcellulose), may also be present. In some embodiments, the compositions are isotonic, i.e., they can have the same osmotic pressure as blood and lacrimal fluid.

In some embodiments, the desired isotonicity of the compositions can be accomplished using sodium chloride, or other pharmaceutically acceptable agents such as dextrose, boric acid, sodium tartrate, propylene glycol or other inorganic or organic solutes. Sodium chloride is preferred particularly for buffers containing sodium ions.

In some embodiments, viscosity of the compositions can be maintained at the selected level using a pharmaceutically acceptable thickening agent. In some embodiments, the thickening agent is methylcellulose. Other suitable thickening agents include, for example, xanthan gum, carboxymethyl cellulose, hydroxypropyl cellulose, carbomer, and the like. The preferred concentration of the thickener will depend upon the agent selected and the desired viscosity. Viscous compositions are normally prepared from solutions by the addition of such thickening agents.

In some embodiments, a pharmaceutically acceptable preservative or cell stabilizer can be employed to increase the life of the compositions. In some embodiments, if preservatives are necessary, it is well within the purview of the skilled artisan to select compositions that will not affect the viability or efficacy of the cells as described in the present invention.

Following transplantation, in some embodiments, the growth or differentiation of the administered cells or the therapeutic effect of the cells can be monitored. For example, blood glucose, serum glucose, HbAlc (a measure of glycosylated protein) and/or serum insulin can be monitored.

Following administration, in some embodiments, the immunological tolerance of the subject to the cells may be tested by various methods known in the art to assess the subject's immunological tolerance to the cells. In cases where the subject's tolerance of the cells is suboptimal (e.g., the subject's immune system is rejecting the exogenous cells), therapeutic adjunct immunosuppressive treatment, which is known in the art, of the subject may be performed.

Application of the teachings of the present invention to a specific problem is within the capabilities of one having ordinary skill in the art in light of the teaching contained herein. Examples of the compositions and methods of the invention appear in the following non-limiting Examples.

### EXAMPLES

### Example 1. Co-Transplantation of MMP-9 Enhanced Mesenchymal Stem Cells and Pancreatic Islets for the Treatment of Type 1 Diabetes

We have genetically modified MSC for the enhanced secretion of certain molecules which are essential for pancreatic beta cell function and islet vascularization. The present example describes a novel cell therapy based on the co-encapsulation of genetically modified MSCs and pancreatic islets to treat diabetic patients. Stromal support with engineered MSCs, including enhanced MMP-9 secretion, further promote engraftment and function of islets after transplantation.

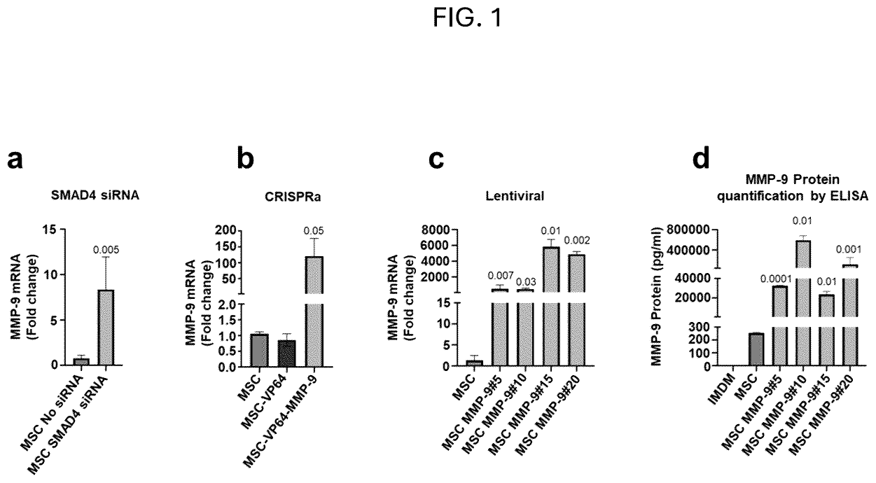

We present a unique and novel stem cell-based therapy in which we combine genetically enhanced MMP9-producing MSCs and insulin-producing pancreatic islets. Described herein are three different methods to upregulate the transcriptional expression of MMP-9 by MSCs. In experiments, we used SMAD4 knockdown using siRNA, CRISPRa/Cas9, and lentivectors, to produce MSCs with ˜5-10-, ˜120-, and ˜1,000-6,000-fold enhanced MMP-9 expression (FIG. 1 a, b, c). We confirmed the increase in secreted MMP-9 protein in the MSC-conditioned media by ELISA (FIG. 1 d).

Both MSCs and islets are co-encapsulated in Calcium-alginate microbeads of 550 μm diameter (FIG. 2) using a clinical-grade BioRep encapsulator.

We will utilize this enhanced live insulin-producing cell therapy product to treat insulin-dependent diabetes. Encapsulated MSC do not produce severe immunological responses when transplanted and function across xenogeneic barriers (Li et al., Xenotransplantation, (2012), 19:273-85). This technology intends to solve an important problem: the poor engraftment of pancreatic islets after intraportal infusion, which drastically reduces the chances of insulin independence. When pancreatic islets are transplanted alone (no encapsulation, no stromal support), up to 75% of them are destroyed by an instant blood-mediated inflammatory reaction (IBMIR) which is characterized by complement activation, platelet, and neutrophil recruitment (Nilsson et al., Current opinion in organ transplantation, (2011), 16:620-6). Our technology addresses both protection against IBMIR (islets are separated from the blood) and promotion of islet engraftment (enhanced MMP-9 secretion). We demonstrated in our previous work that MSCs can improve in vivo and in vitro survival/engraftment and function of encapsulated pancreatic islets by cell-to-cell contact (Montanari, E., et al., Stem Cell Res Ther, (2017), 8:199) (FIG. 3a). MSCs doubled insulin secretion in vitro (FIG. 3b) and encapsulated islets with MSCs reversed diabetes up to 90 days compared to 8 days with unprotected islets and 70 days without MSCs (FIG. 3c). In experiments, we found evidence that MMP-9-enhanced MSCs by lentivectors had further increased stimulation index after glucose challenge (FIG. 3).

### 1. Development of Multiple Genetically Modified MSC Clones Achieving a Robust and Durable MMP-9 Secretion In Vitro.

Human bone marrow-derived MSCs from RoosterBio Inc. will be used for all experiments. Batches of MSCs from multiple different MSC donors will be screened by ELISA to quantify levels of native MMP-9 in their CM to select for our use. We already have established that Smad4 is a down regulator of MMP-9 using siRNA approach. We will then use CRISPR technology to further upregulate the transcriptional expression of MMP-9. In order to obtain a stable source of MMP-9 high-producer MSCs, Smad4 will be targeted for deletion using CRISPR-Cas9 technology. By CRISPRa technology, a nuclease-deficient (“dead”) Cas9 fused to a transactivation domain is delivered by lentivirus to MSCs (Kearns et al., Development, (2014), 141:219-23), along with target-specific sgRNAs to a selected upstream promoter region of MMP-9. As already demonstrated, we have achieved approximately 120-fold upregulation of MMP-9 through CRISPRa technology in one of the MSC clones. Optimal CRIPSRa sgRNAs with no off target effect will be independently determined by screening 4-6 sgRNAs/gene. The ability of each sgRNA, in the presence of the dead Cas9-activator fusion, to stimulate cytokine transcription will be assessed by qRT-PCR. ELISA will be used to monitor the secretion of MMP-9 by unmodified versus CRISPR-engineered MSCs (with human fibroblasts as additional controls) under basal and oxidative stress conditions (hypoxic chamber). Successfully engineered MSCs batches will be cloned, and CRISPR-engineered MSC clones with stable high (≥5-fold increased) secretion of MMP-9 will be selected. Cell division rate and morphology of the best clones will be assessed over time to assess phenotype stability and the absence of an increased growth rate. Engineered clones of MSCs will be monitored over time by ELISA quantitation of their secretion of MMP-9 into culture supernatants.

### 2. Assess the Impact of Modified MSCs Versus Non-Modified MSCs on Pancreatic Islet Function and Survival In Vitro and In Vivo.

### In Vitro Assessment

Islet will be maintained in culture using CMRL media supplemented with human albumin and human serum. We will assess islet insulin secretion by measuring glucose-stimulated insulin secretion (GSIS) and islet perifusion (Alcazar et al., Front Endocrinol (Lausanne), (2019), 10:680) in the following groups:
1) Non-encapsulated islets
2) Encapsulated islets alone
3) Encapsulated islets and non-modified MSCs
4) Encapsulated islets and high MMP-9 secretion MSCs (including subgroups with modified MSCs using the different methods as noted above)

Pancreatic islets will be isolated from human pancreas as previously described, according to an adaptation of Ricordi's semi-automated technique (Ricordi et al., Diabetes, (1988), 37:413-20; Bucher et al., Transplantation, (2005), 79:91-7). Briefly, Collagenase NB2 (Serva Electrophoresis, Heidelberg, Germany) and neural protease will be injected via the pancreatic duct, and the pancreas will be digested in a Ricordi chamber at 37° C. for approximately 15 minutes. Digestion rate will be defined as 100×(pancreas weight−remnant weight)/pancreas weight (%). Islets were purified on a continuous Biocoll gradient (Biochrom, Berlin, Germany) using a refrigerated COBE cell processor (COBE 2991; Cobe, Lakewood, CO). The purity of islets will be assessed by dithizone, viability/cell death will be assessed by FDA/PI staining, and numbers will be counted using metamorph software. A clean preparation of human islets will be prepared and encapsulated with genetically modified third-party MSCs.

We will assess islet morphology and viability over time using paraffin histology and FDA/PI.

### In Vivo Assessment

The functional effect of encapsulated genetically modified MSCs and islets will be further assessed in vivo. We will transplant encapsulated islets intraperitoneally (ip) in diabetic C57BL/6 mice (Montanari, E., et al., Stem Cell Res Ther, (2017), 8:199). Diabetes will be induced in mice by intraperitoneal injection of streptozotocin (STZ) at 220 mg/kg.

The following mice groups will be analyzed:
1) A control group receiving no STZ (n=12)
2) Mice receiving STZ and ip non-encapsulated islets (n=24)
3) Mice receiving STZ and ip encapsulated islets alone (n=24)
4) Mice receiving STZ/and ip encapsulated islets+non-engineered MSCs (n=24) 5) Mice receiving STZ and ip encapsulated islets+high MMP-9 secretion MSCs (n=24)

Diabetes will be defined as a blood glucose level consistently >20 nmol/L. Three days post-injection of streptozotocin, diabetic mice will be transplanted with 3000-4000 IEQ encapsulated islets with or without MSC/modified MSCs will be transplanted ip through a minimally-invasive abdominal incision. Blood glucose will be measured daily for the first-week post-transplantation and twice weekly thereafter. Blood glucose levels of 11 mmol/L on 2 consecutive days will define successful islet function. Islet graft failure will be considered if the glucose level reaches >20 nmol/L for two days. Islet graft survival via C-Peptide measurement will also be performed. Physiological response to glucose challenge will be assessed by intraperitoneal glucose tolerance test (IPGTT). Based on our prior experience, and despite the use of immunocompetent mice, we do not expect significant immune response given the immune protection provided with microcapsules (pores <140 kD allowing small molecule transit only such as insulin, glucose and O2 only) and the immunomodulation by MSCs. At the end of follow-up, in mice with functioning grafts, capsules will be removed from the intraperitoneal cavity to demonstrate a return to a hyperglycemic state. Graft histology will be performed to assess for islet morphology, including staining for β-cells insulin, a-cells glucagon, and 8-cell somatostatin. CD31 will be assessed for neovascularization. Masson Trichrome will be used to determine perigraft collagen deposition. Power: we anticipate that 95% of the mice will reverse diabetes at one month in the islet/MSC group versus 60% in the islet alone group and 0% in the non-encapsulated islets group. With an alpha of 0.05 and a power of 80%, 24 mice per group should be sufficient to reach significance.

We aim to provide a cell therapy product in the form of one to three injections per patient of encapsulated islets with MMP-9 enhanced MSCs. The goal is to prevent severe hypoglycemic episodes, to significantly improve glycemic control, and quality/quantity of life. On Jun. 28, 2023, FDA approved the first allogenic (donor) pancreatic islet cellular therapy for the treatment of type I diabetes. However, the maximal insulin-independence rate at 5-year is 25% to 50%. The present technology will significantly increase that number and develop into a widely used therapeutic regimen for T1D patients. Of note, the data generated in this Example has the potential to allow treatment providers to reduce the number of islet infusions needed for T1D patients, therefore, reducing the costs for third-party payers.

### Example 2. Genetically Modified Mesenchymal Stem Cells for the Treatment Type 1 Diabetes

Mesenchymal stem cells (MSCs) are multipotent adult stem cells found in various tissues such as bone marrow, adipose tissues, and umbilical cord blood. MSCs have immunomodulatory effects, low immunogenicity, and can function across xenogeneic barriers.

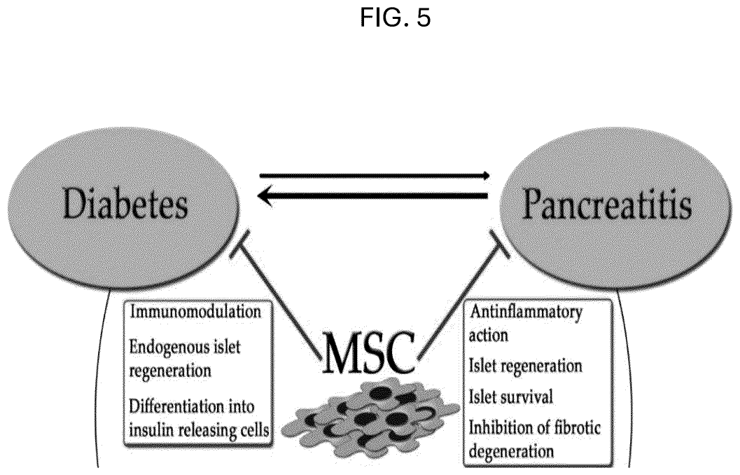

Our focus is to develop mesenchymal stem cells with enhanced function to combat diabetes. We selected MMP-9 for its pro-angiogenic/pro-engraftment role after pancreatic islet transplant. (FIG. 5 and FIG. 1)

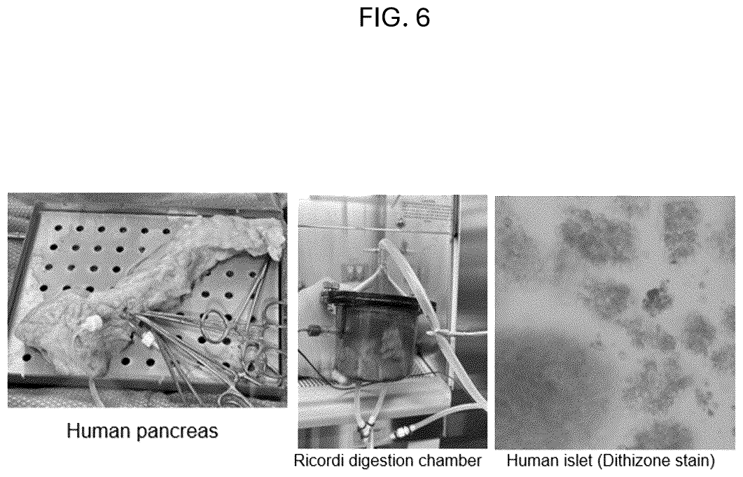

To enhance the MMP-9 production, bone-marrow derived MSC (commercially available from RoosterBio Inc.) were modified using three different methods: (i) SMAD4 silencing, (ii) CRISPRa/dCas9 activation, and (iii) Lentiviral mediated over-expression. Upregulation of MMP-9 was confirmed by qRT-PCR, FACS, and ELISA up to >4000-fold. We tested the effect of MSCs in vitro on pancreatic Beta cell function and survival. See FIG. 3. Human islet isolation is shown in FIG. 6.

We also demonstrated that MSCs (non-modified) can improve insulin secretion and survival/engraftment and function of encapsulated pancreatic islets by cell-to-cell contact. We will test the effect of MMP-9 enhanced MSCs on human islets of Langerhans in vitro and in vivo in diabetic mice using MSCs-islets co-encapsulation.

### Example 3. Xenotransplantation of Encapsulated Human MMP-9 Enhanced Mesenchymal Stem Cells and Pancreatic Islets in Diabetic Mice

Diabetes is one of the most prevalent diseases globally, demanding lifelong insulin dependence with risks including hypoglycemia and kidney complications. Encapsulated pancreatic islet xenotransplant represents hope to broadly treat patients with diabetes and end-stage renal disease (in conjunction with a kidney xenotransplant) and patients with hypoglycemia unawareness. However, limited engraftment of xenogeneic islets remains a barrier. Matrix metalloproteinase (MMP), a key molecule secreted by mesenchymal stem cells (MSCs), has been shown to enhance beta cell function and islet vascularization. The present example tested whether encapsulated human MMP-enhanced MSCs and islets can reverse diabetes in mice.

### Methods

MMP9 overexpression was achieved in MSCs by two different approaches, GM-MSC1 and GM-MSC2, with 6,000-fold and 15-fold increase in MMP, respectively. In this example, the GM-MSCI approach utilized a lentiviral vector encoding MMP, while the GM-MSC2 approach utilized SMAD4 knockdown to achieve MMP overexpression.

The MMP-9 expressed by a lentivirus was provided commercially by GenTarget Inc. Sec, e.g., SKU: LVP395. The SMAD4 knockdown siRNA was provided commercially by ThermoFisher (assay ID s8405; catalog #4390824).

Overexpression of MMP in modified MSCs was confirmed by qPCR and ELISA. We studied the effects of MSCs and modified MSCs on pancreatic islet function and survival in vitro and in vivo. Islet Insulin secretion was evaluated in vitro by measuring glucose-stimulated insulin secretion (GSIS). Islet morphology and viability were assessed over time using FDA/PI staining. Diabetes was induced in C57BL/6 mice by intraperitoneal injection of streptozotocin (STZ) at 220 mg/kg. Diabetes was defined as glycemia levels >360 mg/dL. Encapsulated human islets were xenotransplanted intraperitoneally in diabetic mice within groups: islets alone, islets+MSCs, islets+GM-MSC1, and islets+GM-MSC2. Blood glucose level was monitored twice a week for each mice group. Islet xenograft failure was concluded when glucose level was >360 mg/dL for three consecutive measurements.

For encapsulation, 3.2% of sodium alginate (purchased from Noavamatrix-4200101, Na-Alginate UP-MVG) was prepared in 1× phosphate buffer saline (PBS). An equal volume of 3.2% sodium alginate solution was mixed with an equal volume of non-modified MSCs or GM-MSCs (at a concentration of 1.5 million cells per ml). A mix of sodium alginate and MSCs/GM-MSCs solutions was loaded in an encapsulator (purchased from Biorep Inc, CE-01) and passed through the encapsulator using co-axial air flow at 0.3 ml/min, 3 LPM pressure. Capsules were collected in a stirring calcium bath (10 mM MOPS+100 mM Calcium chloride di hydrate, 1.5 mM DTT). Collected capsules (approx size 500 μM) were washed with 1×PBS and were transplanted in mice or stored them in IMDM medium supplemented with 10% FBS in 5% CO2 incubator.

### Results

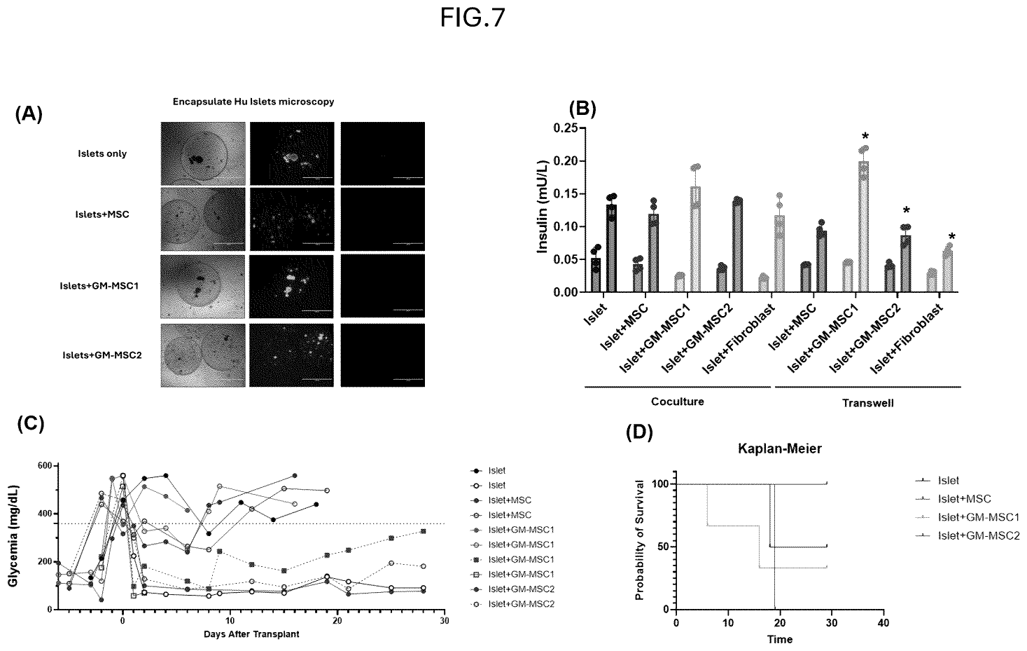

FDA/PI staining assessed islet viability post-encapsulation, indicating excellent cell viability (FIG. 7A) in all the groups. Islets co-cultured with GM-MSCI demonstrated a significantly increased (p=0.02) insulin response compared to the islets alone or other groups (FIG. 7B). Streptozotocin-induced diabetic mice were transplanted with encapsulated islets (n=2), encapsulated islets+MSCs (n=2), encapsulated islets+GM-MSC1 (n=4), and islets+GM-MSC2 (n=2). Islet+GM-MSC2 and GM-MSC1 showed lower blood glucose than unmodified MSCs (FIG. 7C). GM-MSC2 demonstrated improved xenograft survival and prevented mouse mortality (FIG. 7D). This phenomenon could result from the microenvironment provided by GM-MSC2, which promotes beta cell function and improves xenograft vascularization.

### CONCLUSIONS

The present study shows that MSCs with upregulated MMP expression improved the survival and function of microencapsulated human islets when transplanted into mice. The data suggests that MMP secreted by MSCs and their microenvironment improve beta cell function in response to glucose stimulation.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

1. A cellular therapeutic composition for treating diabetes in a subject comprising:
i) a therapeutically effective amount of insulin producing cells; and
ii) a therapeutically effective amount of modified mesenchymal stem cells, wherein the mesenchymal stem cells have been modified to increase expression of matrix metalloproteinase-9 (MMP-9) or a fragment or variant thereof.

2. The cellular therapeutic composition of claim 1, wherein the insulin producing cells are pancreatic cells.

3. The cellular therapeutic composition of claim 2, wherein the pancreatic cells are islet cells.

4. The cellular therapeutic composition of claim 3, wherein the islet cells are β-cells.

5. The cellular therapeutic composition of claim 1, wherein the insulin producing cells are differentiated from stem cells.

6-12. (canceled)

13. The cellular therapeutic composition of claim 1, wherein the mesenchymal stem cells have been modified to increase expression of MMP-9 or a fragment or variant thereof by administering to the mesenchymal stem cells a nucleic acid that encodes MMP-9 or a fragment or variant thereof.

14. (canceled)

15. (canceled)

16. The cellular therapeutic composition of claim 1, wherein the mesenchymal stem cells have been modified to increase expression of MMP-9 by reducing expression of SMAD4.

17-20. (canceled)

21. The cellular therapeutic composition of claim 1, wherein the insulin producing cells and modified mesenchymal stem cells are co-encapsulated.

22. The cellular therapeutic composition of claim 21, wherein the encapsulated cells are immobilized within a polymeric semi-permeable scaffold.

23. The cellular therapeutic composition of claim 1, wherein the insulin producing cells and modified mesenchymal stem cells are encapsulated by a semi-permeable scaffold comprising alginate.

24. The cellular therapeutic composition of claim 23, wherein the semi-permeable scaffold comprises calcium and alginate.

25. The cellular therapeutic composition of claim 24, wherein the insulin producing cells and modified mesenchymal stem cells are co-encapsulated in calcium-alginate microbeads of about 550 μm diameter.

26. A method of treating or preventing diabetes in a subject, comprising administering to the subject an effective amount of the cellular therapeutic composition of claim 1.

27. The method of claim 26, wherein the subject has Type I diabetes.

28. The method of claim 26, wherein the subject has Type II diabetes.

29. The method of claim 26, wherein the cellular therapeutic composition is administered to the subject by infusion through the subject's portal vein.

30. The method of claim 26, wherein the cellular therapeutic composition is administered to the subject intraperitoneally.

31. The method of claim 26, wherein the insulin producing cells are engrafted in the subject following administration.

32. The method of claim 26, wherein the insulin producing cells secrete an effective amount of insulin in response to the subject's blood glucose levels, thereby lowering blood glucose levels in the subject and treating diabetes.


SEQUENCE LISTING (6 sequences)

SEQ ID NO: 1 - protein, 707 aa, Homo sapiens
MSLWQPLVLVLLVLGCCFAAPRQRQSTLVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGVVVPTRFGNADGAACHFPFIFEGRSYSACTTDGRSDGLPWCSTTANYDTDDRFGFCPSERLYTRDGNADGKPCQFPFIFQGQSYSACTTDGRSDGYRWCATTANYDRDKLFGFCPTRADSTVMGGNSAGELCVFPFTFLGKEYSTCTSEGRGDGRLWCATTSNFDSDKKWGFCPDQGYSLFLVAAHEFGHALGLDHSSVPEALMYPMYRFTEGPPLHKDDVNGIRHLYGPRPEPEPRPPTTTTPQPTAPPTVCPTGPPTVHPSERPTAGPTGPPSAGPTGPPTAGPSTATTVPLSPVDDACNVNIFDAIAEIGNQLYLFKDGKYWRFSEGRGSRPQGPFLIADKWPALPRKLDSVFEEPLSKKLFFFSGRQVWVYTGASVLGPRRLDKLGLGADVAQVTGALRSGRGKMLLFSGRRLWRFDVKAQMVDPRSASEVDRMFPGVPLDTHDVFQYREKAYFCQDRFYWRVSSRSELNQVDQVGYVTYDILQCPED

SEQ ID NO: 2 - DNA, 2124 bp, Homo sapiens
  source 1..2124: mol_type = other DNA
atgagcctctggcagcccctggtcctggtgctcctggtgctgggctgctgctttgctgcccccagacagcgccagtccacccttgtgctcttccctggagacctgagaaccaatctcaccgacaggcagctggcagaggaatacctgtaccgctatggttacactcgggtggcagagatgcgtggagagtcgaaatctctggggcctgcgctgctgcttctccagaagcaactgtccctgcccgagaccggtgagctggatagcgccacgctgaaggccatgcgaaccccacggtgcggggtcccagacctgggcagattccaaacctttgagggcgacctcaagtggcaccaccacaacatcacctattggatccaaaactactcggaagacttgccgcgggcggtgattgacgacgcctttgcccgcgccttcgcactgtggagcgcggtgacgccgctcaccttcactcgcgtgtacagccgggacgcagacatcgtcatccagtttggtgtcgcggagcacggagacgggtatcccttcgacgggaaggacgggctcctggcacacgcctttcctcctggccccggcattcagggagacgcccatttcgacgatgacgagttgtggtccctgggcaagggcgtcgtggttccaactcggtttggaaacgcagatggcgcggcctgccacttccccttcatcttcgagggccgctcctactctgcctgcaccaccgacggtcgctccgacggcttgccctggtgcagtaccacggccaactacgacaccgacgaccggtttggcttctgccccagcgagagactctacacccgggacggcaatgctgatgggaaaccctgccagtttccattcatcttccaaggccaatcctactccgcctgcaccacggacggtcgctccgacggctaccgctggtgcgccaccaccgccaactacgaccgggacaagctcttcggcttctgcccgacccgagctgactcgacggtgatggggggcaactcggcgggggagctgtgcgtcttccccttcactttcctgggtaaggagtactcgacctgtaccagcgagggccgcggagatgggcgcctctggtgcgctaccacctcgaactttgacagcgacaagaagtggggcttctgcccggaccaaggatacagtttgttcctcgtggcggcgcatgagttcggccacgcgctgggcttagatcattcctcagtgccggaggcgctcatgtaccctatgtaccgcttcactgaggggccccccttgcataaggacgacgtgaatggcatccggcacctctatggtcctcgccctgaacctgagccacggcctccaaccaccaccacaccgcagcccacggctcccccgacggtctgccccaccggaccccccactgtccacccctcagagcgacccacagctggccccacaggtcccccctcagctggccccacaggtccccccactgctggcccttctacggccactactgtgcctttgagtccggtggacgatgcctgcaacgtgaacatcttcgacgccatcgcggagattgggaaccagctgtatttgttcaaggatgggaagtactggcgattctctgagggcagggggagccggccgcagggccccttccttatcgccgacaagtggcccgcgctgccccgcaagctggactcggtctttgaggagccgctctccaagaagcttttcttcttctctgggcgccaggtgtgggtgtacacaggcgcgtcggtgctgggcccgaggcgtctggacaagctgggcctgggagccgacgtggcccaggtgaccggggccctccggagtggcagggggaagatgctgctgttcagcgggcggcgcctctggaggttcgacgtgaaggcgcagatggtggatccccggagcgccagcgaggtggaccggatgttccccggggtgcctttggacacgcacgacgtcttccagtaccgagagaaagcctatttctgccaggaccgcttctactggcgcgtgagttcccggagtgagttgaaccaggtggaccaagtgggctacgtgacctatgacatcctgcagtgccctgaggactag

SEQ ID NO: 3 - protein, 120 aa, synthetic construct
HHISGLYGLUGLYTHRPHETHRSERASPLEUSERLYSGLNMETGLUGLUGLUALAVALARGLEUPHEILEGLUTRPLEULYSASNGLYGLYPROSERSERGLYALAPROPROPROSERNH

SEQ ID NO: 4 - RNA, 20 nt, synthetic construct
  source 1..20: mol_type = other RNA
aactctgtacaaagaccgcg

SEQ ID NO: 5 - RNA, 20 nt, synthetic construct
  source 1..20: mol_type = other RNA
ttcttcctaaggttgcacat

SEQ ID NO: 6 - RNA, 20 nt, synthetic construct
  source 1..20: mol_type = other RNA
gcagtggagagaggaggagg